(19) United States
(12) Patent Application Publication
(10) Pub. No.: US 2021/0145654 A1
(43) Pub. Date: May 20, 2021

(54) DIFFERENTIAL RFID MOISTURE SENSING SYSTEM

(71) Applicant: Pixie Scientific, LLC, New York, NY (US)

(72) Inventors: Scott MEEK, Watchung, NJ (US); William GOTIMER, Richwood, NY (US); Daniel HOWARD, Brooklyn, NY (US)

(21) Appl. No.: 17/099,461

(22) Filed: Nov. 16, 2020

Related U.S. Application Data
(60) Provisional application No. 62/936,007, filed on Nov. 15, 2019.

(51) Int. Cl. A61F 13/42; G06K 7/10; G06K 19/077; H04B 5/00

(52) U.S. Cl. CPC A61F 13/42; G06K 7/10366; A61F 2013/15121; H04B 5/0062; A61F 2013/424; G06K 19/0772

(57) ABSTRACT

A moisture-sensing system configured for sensing moisture in an incontinence product includes two or more UHF RFID tags disposed on and/or within an incontinence product such as a diaper, pad, and/or other suitable device. The UHF RFID tags are configured to transmit differentially based on a presence, amount, and/or spatial extent of moisture in the incontinence product, such that information about a wetness status of the incontinence product can be determined based on which tags produce a signal. The UHF RFID tags include at least one tag configured not to produce a signal in a wet environment, such that the failure of the tag to produce a signal indicates the presence of moisture in proximity to the tag.

### CROSS-REFERENCES

The following applications and materials are incorporated herein, in their entireties, for all purposes: U.S. Provisional Patent Application Ser. No. 62/936,007, filed Nov. 15, 2019.

### FIELD

This disclosure relates to systems and methods for sensing wetness in incontinence products using RFID systems.

### INTRODUCTION

A diaper or other incontinence product should be changed when wet to reduce the wearer's discomfort and risk of skin rash or other problems. Accordingly, it is beneficial to be able to check for and/or monitor wetness in an incontinence product quickly and easily. However, wetness detection systems face several challenges. For example, a wetness detection system must be usable when sensors of the system are in proximity to moisture (e.g., urine and/or other material discharged from the body). Additionally, if the incontinence product is disposable, any sensors that are disposed of along with the used incontinence product must be relatively inexpensive, or else the wetness detection system may be prohibitively costly. Better solutions are needed for sensing wetness of incontinence products.

### SUMMARY

The present disclosure provides systems, apparatuses, and methods relating to moisture-sensing systems.

In some embodiments, a method for detecting moisture in an incontinence product includes: interrogating, using an RFID reader, a first UHF RFID tag and a second UHF RFID tag, each of the first and second UHF RFID tags being coupled to an incontinence product comprising an absorbent material; in response to affirmative signals from the first and the second RFID tags, determining that the absorbent material is dry; and in response to an affirmative signal from only the first RFID tag, determining that the absorbent material is wet.

In some embodiments, a method for detecting moisture in an incontinence product includes: interrogating, using an RFID reader, a first RFID tag and a second RFID tag, wherein the first and second RFID tags are included in the incontinence product; and in response to an affirmative signal from only the first RFID tag, communicating an alert indicating that an absorbent material of the incontinence product is wet.

In some embodiments, a method for assessing wetness of an incontinence product including two or more RFID tags includes: interrogating, using an RFID reader, the two or more RFID tags of the incontinence product; and in response to affirmative signals from a number of the RFID tags, determining information about the wetness of the incontinence product.

Features, functions, and advantages may be achieved independently in various embodiments of the present disclosure, or may be combined in yet other embodiments, further details of which can be seen with reference to the following description and drawings.

### BRIEF DESCRIPTION OF THE DRAWINGS

FIG. 1 is a schematic diagram of an illustrative moisture-sensing system in accordance with aspects of the present teachings.

FIG. 2 is a top view of an illustrative incontinence product including a moisture-sensing configuration of RFID tags, in accordance with aspects of the present teachings.

FIG. 3 is a schematic diagram of another illustrative moisture-sensing system in accordance with aspects of the present teachings.

FIG. 4 is a schematic diagram of an illustrative moisture-monitoring system in accordance with aspects of the present teachings.

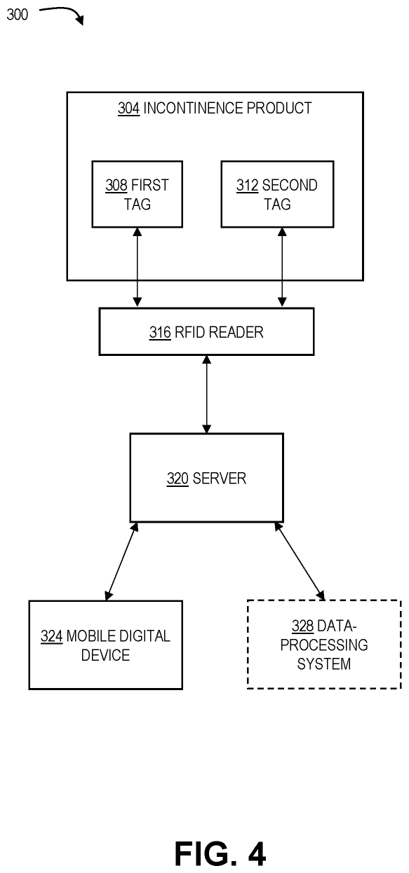

FIG. 5 is a flow chart depicting steps of an illustrative method for detecting moisture in an incontinence product using RFID tags, in accordance with aspects of the present teachings.

FIG. 6 is a schematic diagram of an illustrative data processing system suitable for use with aspects of the present disclosure.

FIG. 7 is a schematic diagram of a computer network suitable for use with aspects of the present disclosure.

### DETAILED DESCRIPTION

Various aspects and examples of a wetness detection system using UHF RFID tags, as well as related methods, are described below and illustrated in the associated drawings. Unless otherwise specified, a wetness detection system in accordance with the present teachings, and/or its various components, may contain at least one of the structures, components, functionalities, and/or variations described, illustrated, and/or incorporated herein. Furthermore, unless specifically excluded, the process steps, structures, components, functionalities, and/or variations described, illustrated, and/or incorporated herein in connection with the present teachings may be included in other similar devices and methods, including being interchangeable between disclosed embodiments. The following description of various examples is merely illustrative in nature and is in no way intended to limit the disclosure, its application, or uses. Additionally, the advantages provided by the examples and embodiments described below are illustrative in nature and not all examples and embodiments provide the same advantages or the same degree of advantages.

This Detailed Description includes the following sections, which follow immediately below: (1) Definitions; (2) Overview; (3) Examples, Components, and Alternatives; (4) Advantages, Features, and Benefits; and (5) Conclusion. The Examples, Components, and Alternatives section is further divided into subsections, each of which is labeled accordingly.

### Definitions

The following definitions apply herein, unless otherwise indicated.

“Substantially” means to be more-or-less conforming to the particular dimension, range, shape, concept, or other aspect modified by the term, such that a feature or component need not conform exactly. For example, a “substantially cylindrical” object means that the object resembles a cylinder, but may have one or more deviations from a true cylinder.

“Comprising,” “including,” and “having” (and conjugations thereof) are used interchangeably to mean including but not necessarily limited to, and are open-ended terms not intended to exclude additional, unrecited elements or method steps.

Terms such as “first”, “second”, and “third” are used to distinguish or identify various members of a group, or the like, and are not intended to show serial or numerical limitation.

“AKA” means “also known as,” and may be used to indicate an alternative or corresponding term for a given element or elements.

“Coupled” means connected, either permanently or releasably, whether directly or indirectly through intervening components.

“Processing logic” means any suitable device(s) or hardware configured to process data by performing one or more logical and/or arithmetic operations (e.g., executing coded instructions). For example, processing logic may include one or more processors (e.g., central processing units (CPUs) and/or graphics processing units (GPUs)), microprocessors, clusters of processing cores, FPGAs (field-programmable gate arrays), artificial intelligence (AI) accelerators, digital signal processors (DSPs), and/or any other suitable combination of logic hardware.

“Providing,” in the context of a method, may include receiving, obtaining, purchasing, manufacturing, generating, processing, preprocessing, and/or the like, such that the object or material provided is in a state and configuration for other steps to be carried out.

In this disclosure, one or more publications, patents, and/or patent applications may be incorporated by reference. However, such material is only incorporated to the extent that no conflict exists between the incorporated material and the statements and drawings set forth herein. In the event of any such conflict, including any conflict in terminology, the present disclosure is controlling.

### Overview

In general, a moisture-sensing system in accordance with aspects of the present teachings may include an incontinence product including two or more radio frequency identification (RFID) tags configured to transmit differentially based on the presence of moisture in the incontinence product.

In some examples, the incontinence product includes a first RFID tag configured to transmit irrespective of proximity to moisture (e.g., urine and/or any other bodily substance comprising a suitable amount of water), and a second RFID tag configured to transmit when in a dry environment and to fail to transmit when in proximity to moisture (e.g., in a wet environment). Accordingly, both the first and second RFID tags transmit signals if the incontinence product is dry, but only the first RFID tag will transmit a signal if the incontinence product is wet (e.g., wet to a sufficient degree and/or within sufficient proximity to the second tag). A detection system can determine that the product is wet in response to the loss of signal from the second RFID tag.

In some examples, the incontinence product includes two or more RFID tags disposed at different locations on the incontinence product and configured not to transmit signals in proximity to moisture. Accordingly, based on which of the RFID tags are transmitting signals and which are not, the location of moisture in the incontinence product can be determined. In some cases, the incontinence product includes no RFID tags configured to transmit in proximity to moisture. This can reduce the cost of the product, as tags configured to transmit in wet environments can be more costly than standard tags. However, an RFID tag configured to transmit in wet environments may optionally be included, such that both differential tag type and differential tag placement can be utilized to achieve a desired product response. For example, two or more RFID tags configured to fail in a wet environment may be placed at different locations on the incontinence product, such that the spatial extent of moisture in the product can be determined based on which of the tags are transmitting signals, and a control tag configured to continue working (i.e., not to fail) in a wet environment may be placed in any suitable location on the incontinence product.

An example of an RFID tag configured to fail/not to transmit in proximity to moisture is a standard ultra-high frequency (UHF) RFID tag. Water, which has a high dielectric constant, inductively changes the impedance of UHF RFID circuits, such that UHF RFID tags are detuned when in proximity to water. The detuned UHF RFID tags are unable to transmit a signal. On the other hand, some UHF RFID tags are specifically designed to transmit in high-dielectric environments, providing an example of an RFID tag configured to transmit in proximity to moisture.

UHF tags are used in examples described herein rather than low-frequency (LF) or high-frequency (HF) tags, because LF and HF tags are not detuned by the presence of water in the same way that UHF tags are. Another consideration is that the lower read ranges of typical LF and HF tags may make their use impractical. However, in other examples, any suitable readable devices configured to transmit or to fail to transmit in the presence of water as appropriate may be used.

The moisture-sensing system of the present disclosure may further include an RFID reader configured to read the RFID tags (e.g., to receive signals transmitted by the RFID tags). In some examples, the RFID reader is configured to communicate information based on the received signals to a data-processing system, such as a computer and/or smartphone. Based on the received information, the data-processing system may determine that the incontinence product is wet, determine a degree of wetness of the incontinence product, determine a spatial extent of wetness in the incontinence product, and/or determine any other suitable information relating to wetness of the incontinence product.

Alternatively, or additionally, the RFID reader may include and/or be integrated into a device including processing logic configured to determine information including the presence, degree, and/or spatial extent of wetness of the incontinence product, and/or any other suitable information. The device may be configured to communicate the determined information to a data-processing system, to display the determined information on a user interface of the device, to alert a local and/or remote user, and/or take any other suitable action.

In some examples, the RFID reader is configured to communicate data based on the received RFID signals to a server (e.g., a cloud-based server). The server is configured to analyze the data to determine, e.g., a presence, degree, and/or spatial extent of moisture of the incontinence product. The server is further configured to communicate information based on the analyzed data to a data-processing system (e.g., a computer, smartphone, tablet, smartwatch, and/or the like). For example, the data-processing system may be configured to produce an alert perceptible by a caregiver, so that the caregiver may check on a user of the incontinence product. In some examples, the server is further configured to transmit information such as a timestamp (e.g., a time at which the RFID signals were received at the RFID reader, or at which data was received at the server), a location (e.g., information identifying a location of the RFID reader and/or RFID tags), patient-identifying information (e.g., information identifying a user of the incontinence product), and/or the like. In some examples, the server and/or data-processing system are configured to analyze data associated with one or more RFID readers over a selected time interval (e.g., to determine statistics of the data, to identify patterns in the data, to predict future needs, and/or to perform any other suitable analytics).

In some examples, processing logic of the RFID reader, the server, and/or a data-processing system in communication with the reader and/or server is configured to determine whether two or more readings of the RFID tags within a predetermined time interval (e.g., a short time interval) are consistent with each other. For example, if the RFID reader interrogates the RFID tags of the incontinence product at a first time, and then interrogates the RFID tags at a second time within a predetermined interval of the first time, the processing logic may check whether the outcomes of the first and second interrogations are consistent (e.g., both indicating that the product is dry, both indicating that the product is wet, both indicating a same or similar degree and/or spatial extent of wetness, and/or any other suitable measure of consistency). In response to determining that the outcomes are inconsistent, the processing logic may alert the user to a possible malfunction, perform a third interrogation and compare the outcome to the first and/or second outcomes, advise a user to perform a third interrogation, and/or take any other suitable action.

In some examples, the moisture-sensing system is used in a facility having many users of incontinence products, such as a nursery, hospital, or nursing home. The system may include one or more RFID readers configured to transmit, along with information based on RFID tag signals received by the readers, information identifying a location and/or user (e.g., a room number, bed number, patient ID number, etc.) associated with the incontinence product. The server and/or a data-processing system in communication with the server may be configured to perform analytics on information received from the RFID reader(s) to, e.g., identify patterns of incontinence product use in the facility.

Although the illustrative moisture-sensing systems described herein are described in the context of incontinence products, moisture-sensing systems in accordance with aspects of the present teachings may be implemented in any suitable device and/or system wherein moisture detection is desired.

### Examples, Components, and Alternatives

The following sections describe selected aspects of exemplary differential RFID wetness sensors, as well as related systems and/or methods. The examples in these sections are intended for illustration and should not be interpreted as limiting the scope of the present disclosure. Each section may include one or more distinct embodiments or examples, and/or contextual or related information, function, and/or structure.

### A. First Illustrative Moisture-Sensing System

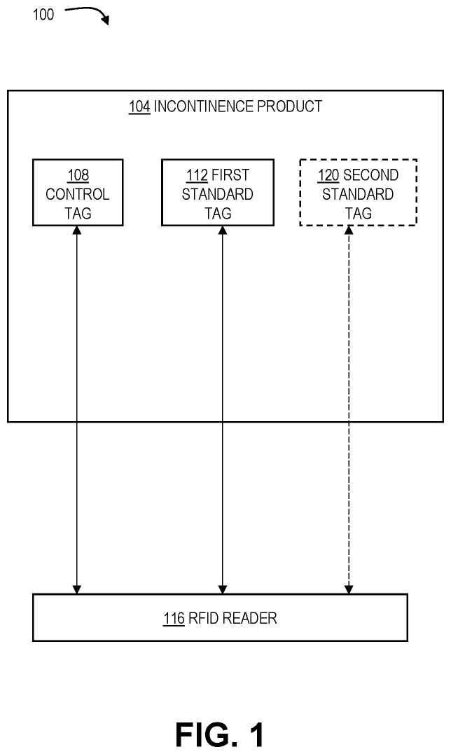

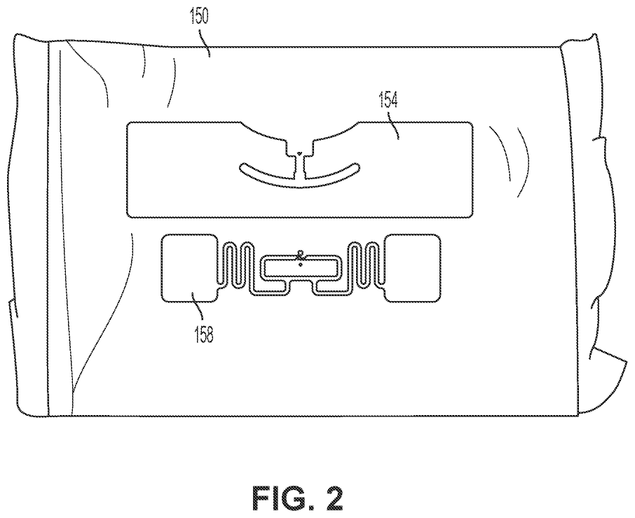

With reference to FIGS. 1-2, this section describes an illustrative moisture-sensing system 100 in accordance with aspects of the present teachings. System 100 is an example of the moisture-sensing systems described above.

FIG. 1 is a schematic diagram of moisture-sensing system 100. System 100 includes an incontinence product 104, which may be a diaper, brief, pullup, insertable pad, bed pad, and/or any other suitable product configured to absorb and/or contain bodily substance(s).

At least one control tag 108 is disposed on and/or within product 104. Control tag 108 comprises a UHF RFID tag configured not to fail (e.g., configured to operate normally) in proximity to water and/or other high-dielectric-constant materials. Accordingly, control tag 108 is configured to produce a readable signal in a wet environment and in a dry environment.

At least a first standard tag 112 is disposed on product 104. Standard tag 112 comprises a standard UHF RFID tag. As is typical for standard UHF RFID tags, standard tag 112 is configured to fail to transmit in a sufficiently wet environment.

Control tag 108 and standard tag 112 may comprise any suitable pair of RFID tags (e.g., of any shape, type, and/or material composition), configured to have a differential response to high dielectric environments (e.g., water/urine). In other words, functionality is achieved when one tag transmits in the presence of water and another does not. Control tag 108 and standard tag 112 collectively comprise a moisture sensor configured to detect moisture in incontinence product 104.

System 100 further includes an RFID reader 116 configured to read signals transmitted by control tag 108 and standard tag 112. RFID reader 116 may be a portable (e.g., handheld) RFID reader, a stationary RFID reader (e.g., coupled to and/or integrated into equipment or other object(s) not moved during ordinary use), and/or may have any other suitable form.

When incontinence product 104 is in a dry state, RFID reader 116 receives signals from both control tag 108 and standard tag 112. However, when product 104 is in a wet state, the moisture detunes standard RFID tag 112. A sufficient amount of moisture in the product detunes standard RFID tag 112 so much that it no longer transmits a signal (e.g., no longer transmits a signal readable by RFID reader 116). Therefore, in a sufficiently wet state, only control tag 108 is visible to RFID reader 116. Accordingly, a reading by reader 116 from control tag 108 in the absence of a reading from standard tag 112 can be understood to indicate wetness of the product (e.g., a positive “wet” reading).

Control tag 108 may be omitted in some examples. However, including control tag 108 allows a control signal to be read when the incontinence product is wet. If control tag 108 is absent, then no signal is produced when the incontinence product is wet, making it more difficult to affirmatively identify (e.g., using a data-processing system or processing logic in communication with the reader) that a “wet” reading has occurred. Another advantage of the control tag is that it facilitates distinction between a situation in which the incontinence product is wet and a situation in which the RFID tags and/or reader are malfunctioning.

Optionally, incontinence product 104 may include at least a second standard UHF RFID tag 120 having detuning properties different from those of first standard tag 112. For example, a first threshold amount of moisture in the presence of which first standard tag 112 fails to transmit may be lower than a second threshold amount of moisture in the presence of which second standard tag 120 fails to transmit. Accordingly, when product 104 is in a dry state, control tag 108, first standard tag 112, and second standard tag 120 each produce signals. When product 104 is in a first wet state above the first threshold but below the second threshold, control tag 108 and second standard tag 120 each produce signals, but first standard tag 112 does not. When product 104 is in a second wet state above the second threshold, control tag 108 produces a signal, but first and second standard tags 108 and 112 do not. In this manner, differential signals produced by first tag 112 and second tag 120 indicate an amount of moisture in product 104 (e.g., a degree of wetness of the product). Incontinence product 104 may include any suitable number of standard UHF RFID tags configured to fail at different respective levels of moisture, allowing a corresponding number of different levels of moisture (e.g., “dry”, “slightly wet”, “moderately wet”, “very wet”, etc.) to be detected.

In some examples, each tag (or groups of tags) on incontinence product 104 is configured to produce a signal containing an identifier that uniquely identifies the tag and/or the incontinence product in which the tags are included. RFID reader 116 is configured to recognize, based on the identifier(s) contained in the signal, that the RFID tags are included in the same incontinence product. This may, for example, allow the RFID reader to correctly read and interpret the differential signal from the tags of the incontinence product even if other tags (e.g., tags of other incontinence products) are within range of the reader.

For example, the RFID tags of incontinence product 104 may be programmed to produce signals containing a same portion of a code (e.g., a same code prefix, suffix, etc.) unique to that instance of product 104. For example, a code prefix may be unique to the tags of a first instance of product 104, with other instances of product 104 each having a different code prefix, such that the RFID reader can easily identify which instance is which. Alternatively, a discrete set of different codes may be distributed among a plurality of incontinence products, such that each code is used by some, but not all, of the plurality of incontinence products. In this pseudo-unique arrangement, it is unlikely that two incontinence products using identical codes will be in use in the same place at the same time.

Continuing this example, in response to receiving an affirmative signal from all of the tags of the incontinence product, the RFID reader and/or associated processing logic may be configured to recognize, based on the unique or pseudo-unique, that the tags correspond to a same incontinence product, and that the incontinence product is currently dry. The reader and/or associated processing logic may be configured to monitor the incontinence product (e.g., actively, by repeatedly interrogating the tags) to determine whether one or more of the tags previously producing a signal including the code or code portion no longer produces a signal in response to the interrogation, indicating that the incontinence product associated with that code has become wet.

In general, the RFID receiver may be configured to check if readings taken within a certain short time interval agree with each other. This can be done for monitoring purposes, as explained above, and/or to detect temporary errors, thereby preventing false alarms, etc. For example, a selected number of sequential readings of a specified nature may be required before the system will make a determination of wetness, dryness, etc.

FIG. 2 is a top view of an illustrative incontinence pad 150, which is an example of incontinence product 104 described above. Incontinence pad 150 includes a control RFID tag 154 configured to transmit in dry and in wet environments, and a standard UHF RFID tag 158 configured to fail to transmit in wet environments. An example of a UHF RFID tag that may be used as control tag 154 is the RFID tag sold under the name “Alien G RFID White Wet Inlay” by Alien Technology®. An example of a standard UHF RFID tag that may be used as standard tag 158 is the RFID tag sold under the name “Alien Short RFID Clear Wet Inlay” by Alien Technology®.

### B. Second Illustrative Moisture-Sensing System

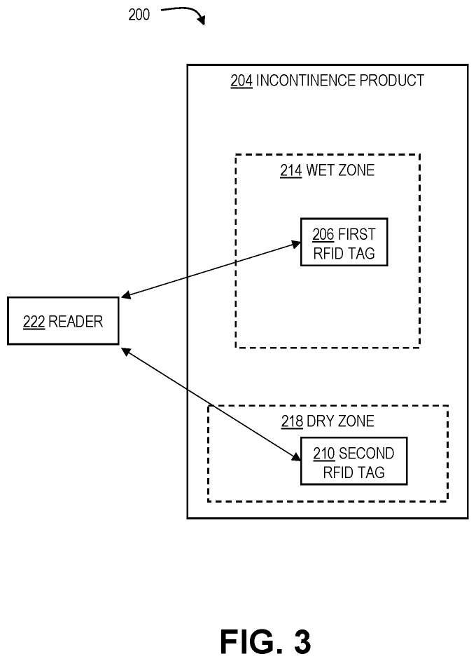

With reference to FIG. 3, this section describes a second illustrative moisture-sensing system 200 in accordance with aspects of the present teachings. System 200 is another example of the moisture-sensing systems described above.

FIG. 3 is a schematic diagram depicting system 200. System 200 includes two or more RFID tags configured to achieve a differential response spatially, by differential tag placement on an incontinence product 204. In the depicted example, system 200 includes a first standard UHF RFID tag 206 and a second standard UHF RFID tag 210. Tags 206 and 210, being standard UHF RFID tags, are each configured to fail to transmit in proximity to water.

First tag 206 is disposed in and/or on a first portion 214 (a “wet zone”) of incontinence product 204 that is expected to become wet when bodily substance(s) are discharged onto product 204 during normal use (e.g., when a user is incontinent). Second tag 210 is disposed in and/or on a second portion 218 (a “dry zone”) of incontinence product 204 that is expected to remain dry during normal use of product 204, even when bodily substance(s) have been discharged onto the product. Second tag 210 is sufficiently far from first portion 214 that moisture in first portion 214 does not prevent second tag 210 from transmitting a signal. This arrangement allows an RFID reader 222 to determine information about the spatial extent of moisture in incontinence product 204. If both first portion 214 and second portion 218 of product 204 are dry, first and second tags 206 and 210 both produce signals. If first portion 214 is wet and second portion 218 is dry, first tag 206 does not produce a signal and second tag 210 does produce a signal. Accordingly, if reader 222 reads signals from both tags, it can be inferred that neither first portion 214 nor second portion 218 is wet. If reader 222 reads signals from second tag 210, but not from first tag 206, it can be inferred that first portion 214 is wet and second portion 218 is dry.

Because second portion 218 does not usually become wet during normal use of the incontinence product, second tag 210 generally produces a signal irrespective of whether the incontinence product is soiled.

In some examples, first RIFD tag 206 is disposed at a location within first portion 214 that typically becomes wet in response to a very small amount of moisture being deposited in the product (e.g., because bodily waste is typically deposited directly onto that location). This allows the moisture-sensing system to alert a user to even a very small amount of moisture in the product, because even a very small amount of moisture will cause the first RFID tag to fail to transmit. In other examples, first RFID tag 206 is disposed at a location within first portion 214 not directly at or adjacent the location where waste is normally directly deposited, such that moisture must spread within the incontinence product to come near enough tag 206 to cause it to fail. In this configuration, the moisture-sensing system tends not to detect moisture until there is enough moisture in the product to spread close enough to tag 206 to cause the tag to fail. In this manner, the sensitivity of the moisture-sensing system can be selected based on the placement of tag 206.

In some examples, two or more standard UHF RFID tags may be disposed at different suitable locations within first portion 214. This allows the system to sense the spread of moisture within the incontinence product based on which tags are transmitting a signal and which have failed. The spread of moisture may indicate an amount of moisture and/or degree of wetness of the incontinence product.

In some examples, more than one standard UHF RFID tag is disposed in second portion 218.

The RFID tags of system 200 may be programmed such that respective signals produced by each tag are configured to uniquely identify the tags as being associated with a common incontinence product.

### C. Illustrative Moisture-Monitoring System

With reference to FIG. 3, this section describes an illustrative moisture-monitoring system 300 in accordance with aspects of the present teachings. Moisture-monitoring system 300 is an example of a system configured to monitor moisture in one or more incontinence products, as described above.

As depicted in FIG. 3, system 300 includes an incontinence product 304 including at least a first RFID tag 308 and a second RFID tag 312. In some examples, tag 308 is configured not to fail in the presence of moisture and tag 312 is configured to fail in the presence of moisture (e.g., as in system 100). In some examples, tag 308 and tag 312 are both configured to fail in the presence of moisture (e.g., as in system 200).

System 300 further includes an RFID reader 316 configured to receive signals transmitted by tags 308 and 312. Reader 316 is a network-connected (e.g., Internet-connected) RFID reader.

Reader 316 is configured to communicate information based on readings of incontinence product 304 (e.g., readings from whichever RFID tags are producing a signal) to a server 320. For example, reader 316 may be configured to communicate read rates for individual tags to be analyzed at server 320, which may be a cloud-based server. As another example, reader 316 may include processing logic configured to determine, based on which tags produce a readable signal, a moisture status of the incontinence product (e.g., “dry” or “wet”, and/or varying degrees of wetness), and the reader may be configured to upload the sensed status to server 320. Alternatively, or additionally, reader 316 may be configured to upload to the server information identifying which tags produce a readable signal, and the moisture status may be determined at the server based on the uploaded information.

Data uploaded to server 320 by reader 316 may be time-stamped, may include information identifying reader 316, may include information identifying incontinence product 304 and/or a product type of product 304, and/or may include any other suitable information. Information identifying reader 316 may be used (e.g., at server 320) to identify a user of incontinence product 304, a room or facility in which the user is located, and/or any other suitable information. Reader 316 may be stationary (e.g., wall-mounted or bed-mounted), portable (e.g., handheld), or wearable (e.g., wrist-mounted). System 300 may include any suitable number of readers 316.

Server 320 has at least two major functions. The first function is to receive the signal from the reader and determine, based on the signal, the wetness status of the incontinence product. This determination may be based on tag read rates, a status signal from the reader, pattern analysis, and/or a combination of these. The status may include “dry” and “wet” and also gradations of “wet”, for example “damp”, “slightly wet”, “saturated”, “very wet”, etc.

The second function of the server is to communicate the incontinence product status to a suitable device and/or person (e.g., a caregiver). This may be accomplished in any suitable manner, e.g., via email, SMS message, notification to a mobile digital device 324 (e.g., a phone, tablet, smartwatch, and/or the like), and/or notification via a web-based or app-based dashboard, which may be accessible via mobile digital device 324 and/or a data-processing system 328 (e.g., a computer).

In addition to, or alternatively to, wetness status and time of status, the notification(s) may include care suggestions to the caregiver, such as “change product soon” or “change product now”. Additionally, patient location, based on reader location and tag identification, may be provided to the caregiver. The care suggestions, patient locations, and/or other suitable information may be determined based on read RFID signals at the server, at the mobile digital device, at the data-processing system, and/or at any other suitable part(s) of system 300.

In some examples, data corresponding to one or more incontinence products by one or more RFID readers is stored (e.g., at server 320) and accessible (e.g., via a software program, web-based app, and/or the like) for analysis.

### D. Illustrative Method

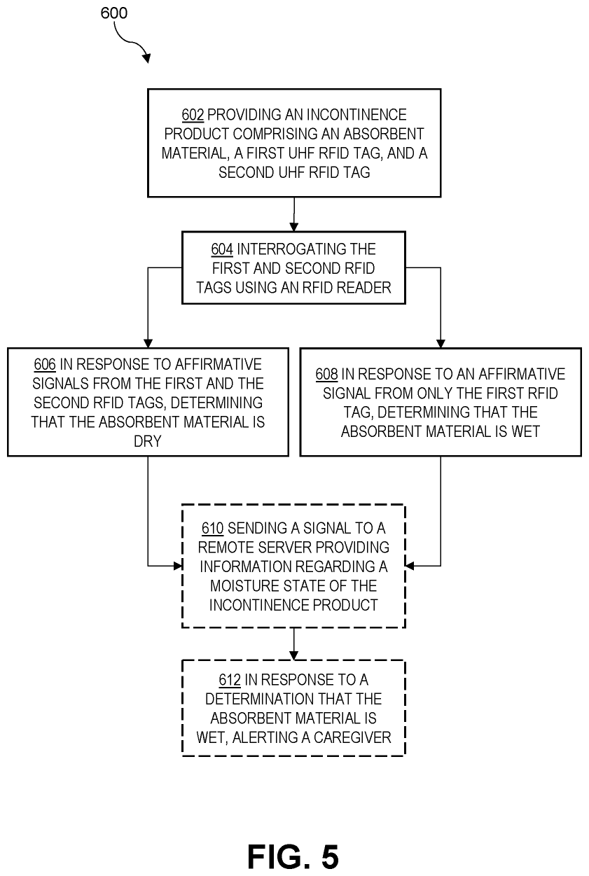

This section describes steps of an illustrative method 600 for wireless wetness monitoring of an incontinence product by differential RFID tag detune; see FIG. 5. Aspects of the systems and sensors described above may be utilized in the method steps described below. Where appropriate, reference may be made to components and systems that may be used in carrying out each step. These references are for illustration, and are not intended to limit the possible ways of carrying out any particular step of the method.

FIG. 5 is a flowchart illustrating steps performed in an illustrative method, and may not recite the complete process or all steps of the method. Although various steps of method 600 are described below and depicted in FIG. 5, the steps need not necessarily all be performed, and in some cases may be performed simultaneously or in a different order than the order shown.

Step 602 of method 600 includes providing an incontinence product comprising an absorbent material, a first UHF RFID tag, and a second UHF RFID tag. In some examples, the first RFID tag is disposed in a first zone of the incontinence product, and the second RFID tag is disposed in a second zone of the incontinence product. In some examples, the first zone corresponds to an area of the incontinence product expected to be dry when a user is incontinent. The second zone may correspond to an area of the absorbent material expected to be wet when a user is incontinent.

Step 604 of method 600 includes interrogating the first and second RFID tags using an RFID reader. In some examples, steps 602 and 604 are combined, and/or the incontinence product may already be present when the method begins.

Step 606 of method 600 includes, in response to affirmative signals from the first and the second RFID tags, determining that the absorbent material is dry. This determination may be implicit or explicit, and may be reflected in a corresponding action taken by processing logic of the RFID reader and/or a server in communication with the RFID reader. In some examples, this step is skipped or inherent, such that the default position of the system is that the absorbent material is dry, unless determined otherwise (see step 608).

Step 608 of method 600 includes, in response to an affirmative signal from only the first RFID tag, determining that the absorbent material is wet. This determination may be implicit or explicit, and may be reflected in a corresponding action taken by processing logic of the RFID reader and/or a server in communication with the RFID reader.

In some examples, the first RFID tag is configured to function in the presence of water. In some examples, the second RFID tag is configured to be detuned by the presence of water. In some examples, both the first RFID tag and the second RFID tag are configured to be detuned by the presence of water.

Optionally, step 610 of method 600 includes, using the RFID reader, sending a signal to a remote server providing information regarding a moisture state of the incontinence product (e.g., wet, dry, etc.).

Optionally, step 612 of method 600 includes, in response to a determination that the absorbent material is wet, alerting a caregiver using the remote server. In some examples, alerting the caregiver comprises automatically sending a text message to an electronic device of the caregiver.

### E. Illustrative Radio-Frequency Identification (RFID) System

Radio-frequency identification (RFID) refers to wireless and typically non-contact use of radio-frequency waves to transfer data. In general, an RFID system includes at least one RFID tag readable by at least one RFID reader. The tag typically includes an integrated circuit (AKA a chip) coupled to a tag antenna. The integrated circuit is configured to modulate a signal such that the signal contains predetermined information including a unique identifier. The tag antenna is configured to transmit the modulated signal (e.g., when interrogated by the reader). The reader is configured to read the tag by receiving the modulated signal using a reader antenna coupled to, or integral with, the reader. Reading the tag in this manner allows the reader to identify the tag based on the data (i.e., the unique identifier) embodied in the modulated signal.

Specific embodiments of an RFID system may be characterized by the frequency of the signal used, on whether the signal to be modulated is generated within the tag or within the reader, on the specific type of electromagnetic coupling between the reader and the tag, and/or on any other suitable factor(s).

For example, an RFID system may include one or more passive tags, semi-passive tags, and/or active tags. A passive RFID tag includes no power source and is configured to receive energy (e.g., through inductive coupling) in the form of radio-frequency waves transmitted by the RFID reader. The received radio-frequency wave is modulated by the circuit of the RFID tag (e.g., via load modulation) and transmitted by the tag's antenna to the reader. In other words, the reader interrogates the passive RFID tag by transmitting a radio-frequency signal to the tag, and then receives a modulated version of the radio-frequency signal.

Like a passive RFID tag, a semi-passive RFID tag is configured to receive a radio-frequency signal from the reader, modulate the signal, and transmit the modulated version of the signal back to the reader. Unlike a passive tag, however, the semi-passive RFID tag does include a power source (e.g., a battery). The power source of the semi-passive RFID tag may provide power to the circuit of the tag, and/or to additional sensors or circuits included in or accompanying the tag. However, the power source does not generate the signal to be transmitted to the reader.

In contrast, an active RFID tag includes a power supply, and uses the power supply to generate a modulated signal to be sent to the reader. In other words, the signal read by the reader originates in the RFID tag, rather than in the reader (as is the case when a passive or semi-passive RFID tag is used). In some examples, an active RFID tag includes additional sensors or circuitry powered by the tag power source.

Some examples of active RFID tags are configured to transmit a modulated signal at a predetermined interval (e.g., every several seconds). An active RFID tag operating in this mode may be referred to as a beacon. Alternatively, or additionally, an active RFID tag may be configured to transmit the modulated signal to the reader in response to receiving a wake-up signal from the reader. A tag operating in this mode may be referred to as an active transponder tag.

RFID systems may be further characterized based on whether they use low-frequency, high-frequency, or ultra-high-frequency radio waves. In an RFID context, the low-frequency (LF) range typically comprises waves having frequencies in the range of 30 kHz to 300 kHz. In some geographic locations, and/or for certain applications, low-frequency RFID systems are required by local laws or rules to operate within a narrower range, such as 125 kHz to 134 kHz, to avoid interference with other signals. Tags of a low-frequency RFID system are typically passive tags configured to couple to an RFID reader via magnetic coupling. Radio waves in the low-frequency range generally propagate through water and/or certain other liquids with little to no absorption. For at least this reason, LF RFID systems may be suitable for applications where the tag or the reader is disposed adjacent a liquid. Typically, LF tags are readable by LF readers disposed at a maximum distance of several centimeters to tens of centimeters, depending on environmental conditions.

High-frequency (HF) RFID systems typically use radio waves having frequencies in the range of 3 MHz to 30 MHz. An HF tag is typically a passive tag powered by and read by an HF reader using inductive coupling. HF RFID systems may have a read range of several centimeters up to approximately a meter. Compared to LF signals, HF signals are more readily absorbed by water and metal. Accordingly, HF systems tend to work poorly in cases where the signal may need to pass through water (or water-based liquids), or through thick layers of metal.

One example of an HF RFID system is a near-field communication (NFC) system, which is a global communication standard operating at 13.56 MHz. In some examples, an NFC device is configured to act as both a reader and a tag (e.g., in a peer-to-peer communication NFC system). Such an NFC device is typically also capable of reading a passive NFC tag, and is in some cases also capable of reading a passive HF RFID tag compliant with suitable communication standards.

Ultra-high frequency (UHF) RFID systems generally operate in the 300 MHz to 3 GHz range. Due to commonly used communication standards and/or regulations, many examples of UHF systems operate either in the range of 860 to 960 MHz, at 433 MHz, or at 2.45 GHz. Some UHF RFID systems are active, and some are passive. Passive UHF RFID tags typically couple to UHF readers electromagnetically via backscatter modulation, rather than the inductive coupling used by typical LF and HF passive systems. One benefit of UHF tags is their range. A passive UHF RFID tag may be readable by a UHF reader at a maximum distance of tens of meters. Some passive UHF tags have ranges of 2-6 meters, for example, sufficient to cover a typical room in a building or home. The read range of an active UHF tag depends on the power of the signal transmitted by the tag, among other factors. Another benefit of UHF tags is that they are generally inexpensive (˜US$0.10).

UHF waves interact readily with liquid and metal (e.g., by reflection, absorption, and/or refraction), and UHF systems therefore typically perform poorly in the presence of these substances unless mitigating techniques or devices are used. Water and other materials having a high dielectric constant inductively change the impedance in UHF tag circuits, detuning them and preventing them from transmitting.

Due to this limitation, manufacturers have begun to produce tags which are designed to mitigate this effect by changing the shape and size of the tag antenna to maximize power absorption as well as tuning the impedance of the tag and chip. Although RFID-based moisture sensors are now commercially available, these sensors are expensive (˜$3 per tag). For example, the RFM2120 moisture sensor is made by RFMicron, Inc. (d/b/a Axzon). Known products use a single, specialized UHF RFID sensor tag that (1) measures the dielectric environment and (2) transmits a sensor code based on that measurement. The user then interprets that sensor code with respect to how close the tag is to moisture. These sensors are more expensive, because they utilize expensive components. In contrast, UHF RFID sensor systems of the present disclosure may have comparable or superior functionality at a fraction of the price. For example, as described above, at least one embodiment of the present disclosure utilizes two standard (e.g., off the shelf) UHF RFID tags, and functions by detecting a differential signal between the two tags.

### F. Illustrative Data Processing System

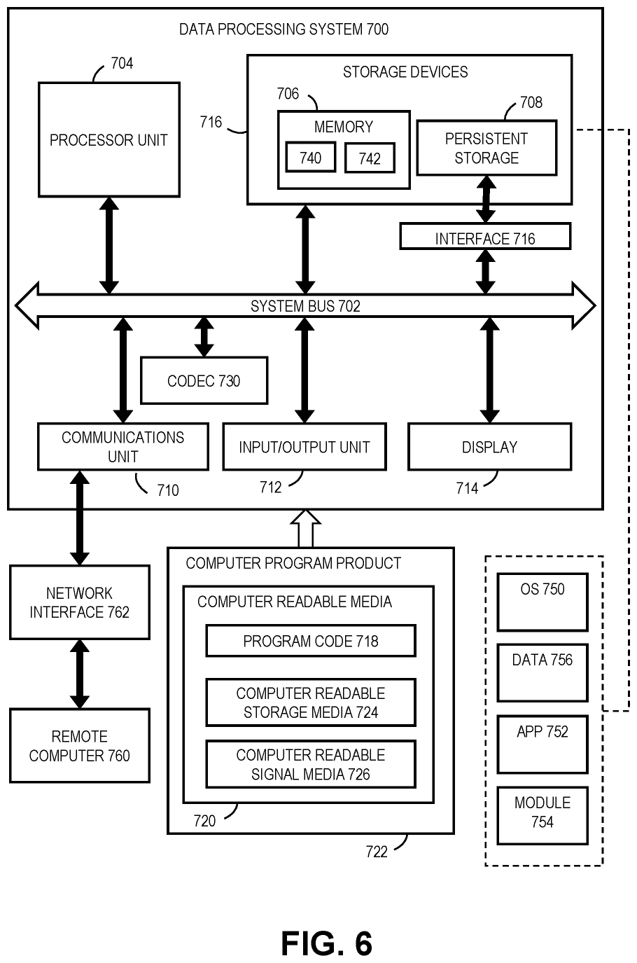

As shown in FIG. 6, this example describes a data processing system 700 (also referred to as a computer, computing system, and/or computer system) in accordance with aspects of the present disclosure. In this example, data processing system 700 is an illustrative data processing system suitable for implementing aspects of the wetness detection system. More specifically, in some examples, the RFID reader, cloud server, and/or the caregiver's electronic device (e.g., smartphone, tablet, personal computer) are embodiments of data processing systems.

In this illustrative example, data processing system 700 includes a system bus 702 (also referred to as communications framework). System bus 702 may provide communications between a processor unit 704 (also referred to as a processor or processors), a memory 706, a persistent storage 708, a communications unit 710, an input/output (I/O) unit 712, a codec 730, and/or a display 714. Memory 706, persistent storage 708, communications unit 710, input/output (I/O) unit 712, display 714, and codec 730 are examples of resources that may be accessible by processor unit 704 via system bus 702.

Processor unit 704 serves to run instructions that may be loaded into memory 706. Processor unit 704 may comprise a number of processors, a multi-processor core, and/or a particular type of processor or processors (e.g., a central processing unit (CPU), graphics processing unit (GPU), etc.), depending on the particular implementation. Further, processor unit 704 may be implemented using a number of heterogeneous processor systems in which a main processor is present with secondary processors on a single chip. As another illustrative example, processor unit 704 may be a symmetric multi-processor system containing multiple processors of the same type.

Memory 706 and persistent storage 708 are examples of storage devices 716. A storage device may include any suitable hardware capable of storing information (e.g., digital information), such as data, program code in functional form, and/or other suitable information, either on a temporary basis or a permanent basis.

Storage devices 716 also may be referred to as computer-readable storage devices or computer-readable media. Memory 706 may include a volatile storage memory 740 and a non-volatile memory 742. In some examples, a basic input/output system (BIOS), containing the basic routines to transfer information between elements within the data processing system 700, such as during start-up, may be stored in non-volatile memory 742. Persistent storage 708 may take various forms, depending on the particular implementation.

Persistent storage 708 may contain one or more components or devices. For example, persistent storage 708 may include one or more devices such as a magnetic disk drive (also referred to as a hard disk drive or HDD), solid state disk (SSD), floppy disk drive, tape drive, Jaz drive, Zip drive, flash memory card, memory stick, and/or the like, or any combination of these. One or more of these devices may be removable and/or portable, e.g., a removable hard drive. Persistent storage 708 may include one or more storage media separately or in combination with other storage media, including an optical disk drive such as a compact disk ROM device (CD-ROM), CD recordable drive (CD-R Drive), CD rewritable drive (CD-RW Drive), and/or a digital versatile disk ROM drive (DVD-ROM). To facilitate connection of the persistent storage devices 708 to system bus 702, a removable or non-removable interface is typically used, such as interface 728.

Input/output (I/O) unit 712 allows for input and output of data with other devices that may be connected to data processing system 700 (i.e., input devices and output devices). For example, input device 732 may include one or more pointing and/or information-input devices such as a keyboard, a mouse, a trackball, stylus, touch pad or touch screen, microphone, joystick, game pad, satellite dish, scanner, TV tuner card, digital camera, digital video camera, web camera, and/or the like. These and other input devices may connect to processor unit 704 through system bus 702 via interface port(s) 736. Interface port(s) 736 may include, for example, a serial port, a parallel port, a game port, and/or a universal serial bus (USB).

Output devices 734 may use some of the same types of ports, and in some cases the same actual ports, as input device(s) 732. For example, a USB port may be used to provide input to data processing system 700 and to output information from data processing system 700 to an output device 734. Output adapter 738 is provided to illustrate that there are some output devices 734 (e.g., monitors, speakers, and printers, among others) which require special adapters. Output adapters 738 may include, e.g., video and sounds cards that provide a means of connection between the output device 734 and system bus 702. Other devices and/or systems of devices may provide both input and output capabilities, such as remote computer(s) 760. Display 714 may include any suitable human-machine interface or other mechanism configured to display information to a user, e.g., a CRT, LED, or LCD monitor or screen, etc.

Communications unit 710 refers to any suitable hardware and/or software employed to provide for communications with other data processing systems or devices. While communication unit 710 is shown inside data processing system 700, it may in some examples be at least partially external to data processing system 700. Communications unit 710 may include internal and external technologies, e.g., modems (including regular telephone grade modems, cable modems, and DSL modems), ISDN adapters, and/or wired and wireless Ethernet cards, hubs, routers, etc. Data processing system 700 may operate in a networked environment, using logical connections to one or more remote computers 760. A remote computer(s) 760 may include a personal computer (PC), a server, a router, a network PC, a workstation, a microprocessor-based appliance, a peer device, a smart phone, a tablet, another network note, and/or the like. Remote computer(s) 760 typically include many of the elements described relative to data processing system 700. Remote computer(s) 760 may be logically connected to data processing system 700 through a network interface 762 which is connected to data processing system 700 via communications unit 710. Network interface 762 encompasses wired and/or wireless communication networks, such as local-area networks (LAN), wide-area networks (WAN), and cellular networks. LAN technologies may include Fiber Distributed Data Interface (FDDI), Copper Distributed Data Interface (CDDI), Ethernet, Token Ring, and/or the like. WAN technologies include point-to-point links, circuit switching networks (e.g., Integrated Services Digital networks (ISDN) and variations thereon), packet switching networks, and Digital Subscriber Lines (DSL).

Codec 730 may include an encoder, a decoder, or both, comprising hardware, software, or a combination of hardware and software. Codec 730 may include any suitable device and/or software configured to encode, compress, and/or encrypt a data stream or signal for transmission and storage, and to decode the data stream or signal by decoding, decompressing, and/or decrypting the data stream or signal (e.g., for playback or editing of a video). Although codec 730 is depicted as a separate component, codec 730 may be contained or implemented in memory, e.g., non-volatile memory 742.

Non-volatile memory 742 may include read only memory (ROM), programmable ROM (PROM), electrically programmable ROM (EPROM), electrically erasable programmable ROM (EEPROM), flash memory, and/or the like, or any combination of these. Volatile memory 740 may include random access memory (RAM), which may act as external cache memory. RAM may comprise static RAM (SRAM), dynamic RAM (DRAM), synchronous DRAM (SDRAM), double data rate SDRAM (DDR SDRAM), enhanced SDRAM (ESDRAM), and/or the like, or any combination of these.

Instructions for the operating system, applications, and/or programs may be located in storage devices 716, which are in communication with processor unit 704 through system bus 702. In these illustrative examples, the instructions are in a functional form in persistent storage 708. These instructions may be loaded into memory 706 for execution by processor unit 704. Processes of one or more embodiments of the present disclosure may be performed by processor unit 704 using computer-implemented instructions, which may be located in a memory, such as memory 706.

These instructions are referred to as program instructions, program code, computer usable program code, or computer-readable program code executed by a processor in processor unit 704. The program code in the different embodiments may be embodied on different physical or computer-readable storage media, such as memory 706 or persistent storage 708. Program code 718 may be located in a functional form on computer-readable media 720 that is selectively removable and may be loaded onto or transferred to data processing system 700 for execution by processor unit 704. Program code 718 and computer-readable media 720 form computer program product 722 in these examples. In one example, computer-readable media 720 may comprise computer-readable storage media 724 or computer-readable signal media 726.

Computer-readable storage media 724 may include, for example, an optical or magnetic disk that is inserted or placed into a drive or other device that is part of persistent storage 708 for transfer onto a storage device, such as a hard drive, that is part of persistent storage 708. Computer-readable storage media 724 also may take the form of a persistent storage, such as a hard drive, a thumb drive, or a flash memory, that is connected to data processing system 700. In some instances, computer-readable storage media 724 may not be removable from data processing system 700.

In these examples, computer-readable storage media 724 is a non-transitory, physical or tangible storage device used to store program code 718 rather than a medium that propagates or transmits program code 718. Computer-readable storage media 724 is also referred to as a computer-readable tangible storage device or a computer-readable physical storage device. In other words, computer-readable storage media 724 is media that can be touched by a person.

Alternatively, program code 718 may be transferred to data processing system 700, e.g., remotely over a network, using computer-readable signal media 726. Computer-readable signal media 726 may be, for example, a propagated data signal containing program code 718. For example, computer-readable signal media 726 may be an electromagnetic signal, an optical signal, and/or any other suitable type of signal. These signals may be transmitted over communications links, such as wireless communications links, optical fiber cable, coaxial cable, a wire, and/or any other suitable type of communications link. In other words, the communications link and/or the connection may be physical or wireless in the illustrative examples.

In some illustrative embodiments, program code 718 may be downloaded over a network to persistent storage 708 from another device or data processing system through computer-readable signal media 726 for use within data processing system 700. For instance, program code stored in a computer-readable storage medium in a server data processing system may be downloaded over a network from the server to data processing system 700. The computer providing program code 718 may be a server computer, a client computer, or some other device capable of storing and transmitting program code 718.

In some examples, program code 718 may comprise an operating system (OS) 750. Operating system 750, which may be stored on persistent storage 708, controls and allocates resources of data processing system 700. One or more applications 752 take advantage of the operating system's management of resources via program modules 754, and program data 756 stored on storage devices 716. OS 750 may include any suitable software system configured to manage and expose hardware resources of computer 700 for sharing and use by applications 752. In some examples, OS 750 provides application programming interfaces (APIs) that facilitate connection of different type of hardware and/or provide applications 752 access to hardware and OS services. In some examples, certain applications 752 may provide further services for use by other applications 752, e.g., as is the case with so-called “middleware.” Aspects of present disclosure may be implemented with respect to various operating systems or combinations of operating systems.

The different components illustrated for data processing system 700 are not meant to provide architectural limitations to the manner in which different embodiments may be implemented. One or more embodiments of the present disclosure may be implemented in a data processing system that includes fewer components or includes components in addition to and/or in place of those illustrated for computer 700. Other components shown in FIG. 6 can be varied from the examples depicted. Different embodiments may be implemented using any hardware device or system capable of running program code. As one example, data processing system 700 may include organic components integrated with inorganic components and/or may be comprised entirely of organic components (excluding a human being). For example, a storage device may be comprised of an organic semiconductor.

In some examples, processor unit 704 may take the form of a hardware unit having hardware circuits that are specifically manufactured or configured for a particular use, or to produce a particular outcome or progress. This type of hardware may perform operations without needing program code 718 to be loaded into a memory from a storage device to be configured to perform the operations. For example, processor unit 704 may be a circuit system, an application specific integrated circuit (ASIC), a programmable logic device, or some other suitable type of hardware configured (e.g., preconfigured or reconfigured) to perform a number of operations. With a programmable logic device, for example, the device is configured to perform the number of operations and may be reconfigured at a later time. Examples of programmable logic devices include, a programmable logic array, a field programmable logic array, a field programmable gate array (FPGA), and other suitable hardware devices. With this type of implementation, executable instructions (e.g., program code 718) may be implemented as hardware, e.g., by specifying an FPGA configuration using a hardware description language (HDL) and then using a resulting binary file to (re)configure the FPGA.

In another example, data processing system 700 may be implemented as an FPGA-based (or in some cases ASIC-based), dedicated-purpose set of state machines (e.g., Finite State Machines (FSM)), which may allow critical tasks to be isolated and run on custom hardware. Whereas a processor such as a CPU can be described as a shared-use, general purpose state machine that executes instructions provided to it, FPGA-based state machine(s) are constructed for a special purpose, and may execute hardware-coded logic without sharing resources. Such systems are often utilized for safety-related and mission-critical tasks.

In still another illustrative example, processor unit 704 may be implemented using a combination of processors found in computers and hardware units. Processor unit 704 may have a number of hardware units and a number of processors that are configured to run program code 718. With this depicted example, some of the processes may be implemented in the number of hardware units, while other processes may be implemented in the number of processors.

In another example, system bus 702 may comprise one or more buses, such as a system bus or an input/output bus. Of course, the bus system may be implemented using any suitable type of architecture that provides for a transfer of data between different components or devices attached to the bus system. System bus 702 may include several types of bus structure(s) including memory bus or memory controller, a peripheral bus or external bus, and/or a local bus using any variety of available bus architectures (e.g., Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small computer systems interface (SCSI)).

Additionally, communications unit 710 may include a number of devices that transmit data, receive data, or both transmit and receive data. Communications unit 710 may be, for example, a modem or a network adapter, two network adapters, or some combination thereof. Further, a memory may be, for example, memory 706, or a cache, such as that found in an interface and memory controller hub that may be present in system bus 702.

The flowcharts and block diagrams described herein illustrate the architecture, functionality, and operation of possible implementations of systems, methods, and computer program products according to various illustrative embodiments. In this regard, each block in the flowcharts or block diagrams may represent a module, segment, or portion of code, which comprises one or more executable instructions for implementing the specified logical function or functions. It should also be noted that, in some alternative implementations, the functions noted in a block may occur out of the order noted in the drawings. For example, the functions of two blocks shown in succession may be executed substantially concurrently, or the functions of the blocks may sometimes be executed in the reverse order, depending upon the functionality involved.

### E. Illustrative Distributed Data Processing System

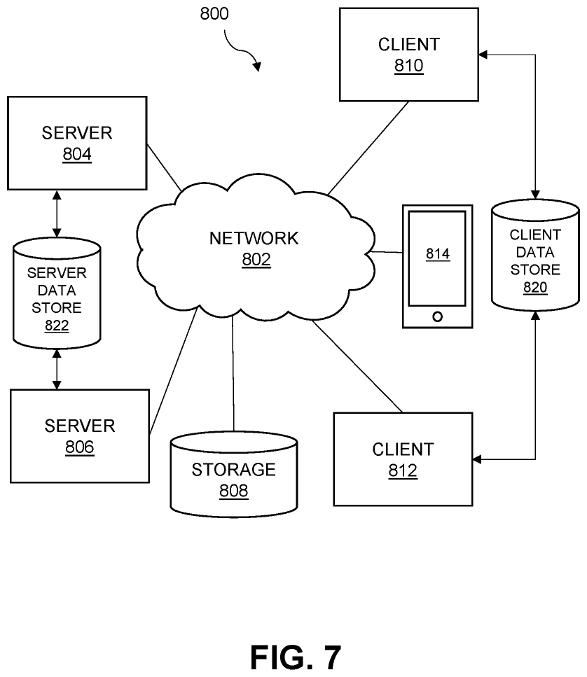

As shown in FIG. 7, this example describes a general network data processing system 800, interchangeably termed a computer network, a network system, a distributed data processing system, or a distributed network, aspects of which may be included in one or more illustrative embodiments of the wetness detection systems described herein. For example, the RFID reader may communicate with the cloud server and the cloud server may communicate with the caregiver's device via one or more computer networks.

It should be appreciated that FIG. 7 is provided as an illustration of one implementation and is not intended to imply any limitation with regard to environments in which different embodiments may be implemented. Many modifications to the depicted environment may be made.

Network system 800 is a network of devices (e.g., computers), each of which may be an example of data processing system 700, and other components. Network data processing system 800 may include network 802, which is a medium configured to provide communications links between various devices and computers connected within network data processing system 800. Network 802 may include connections such as wired or wireless communication links, fiber optic cables, and/or any other suitable medium for transmitting and/or communicating data between network devices, or any combination thereof.

In the depicted example, a first network device 804 and a second network device 806 connect to network 802, as do one or more computer-readable memories or storage devices 808. Network devices 804 and 806 are each examples of data processing system 700, described above. In the depicted example, devices 804 and 806 are shown as server computers, which are in communication with one or more server data store(s) 822 that may be employed to store information local to server computers 804 and 806, among others. However, network devices may include, without limitation, one or more personal computers, mobile computing devices such as personal digital assistants (PDAs), tablets, and smartphones, handheld gaming devices, wearable devices, tablet computers, routers, switches, voice gates, servers, electronic storage devices, imaging devices, media players, and/or other networked-enabled tools that may perform a mechanical or other function. These network devices may be interconnected through wired, wireless, optical, and other appropriate communication links.

In addition, client electronic devices 810 and 812 and/or a client smart device 814, may connect to network 802. Each of these devices is an example of data processing system 700, described above regarding FIG. 6. Client electronic devices 810, 812, and 814 may include, for example, one or more personal computers, network computers, and/or mobile computing devices such as personal digital assistants (PDAs), smart phones, handheld gaming devices, wearable devices, and/or tablet computers, and the like. In the depicted example, server 804 provides information, such as boot files, operating system images, and applications to one or more of client electronic devices 810, 812, and 814. Client electronic devices 810, 812, and 814 may be referred to as “clients” in the context of their relationship to a server such as server computer 804. Client devices may be in communication with one or more client data store(s) 820, which may be employed to store information local to the clients (e,g., cookie(s) and/or associated contextual information). Network data processing system 800 may include more or fewer servers and/or clients (or no servers or clients), as well as other devices not shown.

In some examples, first client electric device 810 may transfer an encoded file to server 804. Server 804 can store the file, decode the file, and/or transmit the file to second client electric device 812. In some examples, first client electric device 810 may transfer an uncompressed file to server 804 and server 804 may compress the file. In some examples, server 804 may encode text, audio, and/or video information, and transmit the information via network 802 to one or more clients.

Client smart device 814 may include any suitable portable electronic device capable of wireless communications and execution of software, such as a smartphone or a tablet. Generally speaking, the term “smartphone” may describe any suitable portable electronic device configured to perform functions of a computer, typically having a touchscreen interface, Internet access, and an operating system capable of running downloaded applications. In addition to making phone calls (e.g., over a cellular network), smartphones may be capable of sending and receiving emails, texts, and multimedia messages, accessing the Internet, and/or functioning as a web browser. Smart devices (e.g., smartphones) may include features of other known electronic devices, such as a media player, personal digital assistant, digital camera, video camera, and/or global positioning system. Smart devices (e.g., smartphones) may be capable of connecting with other smart devices, computers, or electronic devices wirelessly, such as through near field communications (NFC), BLUETOOTH®, WiFi, or mobile broadband networks. Wireless connectively may be established among smart devices, smartphones, computers, and/or other devices to form a mobile network where information can be exchanged.

Data and program code located in system 800 may be stored in or on a computer-readable storage medium, such as network-connected storage device 808 and/or a persistent storage 708 of one of the network computers, as described above, and may be downloaded to a data processing system or other device for use. For example, program code may be stored on a computer-readable storage medium on server computer 804 and downloaded to client 810 over network 802, for use on client 810. In some examples, client data store 820 and server data store 822 reside on one or more storage devices 808 and/or 708.

Network data processing system 800 may be implemented as one or more of different types of networks. For example, system 800 may include an intranet, a local area network (LAN), a wide area network (WAN), or a personal area network (PAN). In some examples, network data processing system 800 includes the Internet, with network 802 representing a worldwide collection of networks and gateways that use the transmission control protocol/Internet protocol (TCP/IP) suite of protocols to communicate with one another. At the heart of the Internet is a backbone of high-speed data communication lines between major nodes or host computers. Thousands of commercial, governmental, educational and other computer systems may be utilized to route data and messages. In some examples, network 802 may be referred to as a “cloud.” In those examples, each server 804 may be referred to as a cloud computing node, and client electronic devices may be referred to as cloud consumers, or the like. FIG. 7 is intended as an example, and not as an architectural limitation for any illustrative embodiments.

### F. Illustrative Combinations and Additional Examples

This section describes additional aspects and features of moisture-sensing systems, presented without limitation as a series of paragraphs, some or all of which may be alphanumerically designated for clarity and efficiency. Each of these paragraphs can be combined with one or more other paragraphs, and/or with disclosure from elsewhere in this application, including the materials incorporated by reference in the Cross-References, in any suitable manner. Some of the paragraphs below expressly refer to and further limit other paragraphs, providing without limitation examples of some of the suitable combinations.

A0. A product comprising any feature described herein, either individually or in combination with any other such feature, in any configuration.

B0. A process for detecting wetness in an incontinence product using RFID tags, the process comprising any process step described herein, in any order, using any modality.

C0. A method for detecting moisture in an incontinence product, the method comprising:

providing an incontinence product comprising an absorbent material, a first UHF RFID tag, and a second UHF RFID tag;

interrogating the first and second RFID tags using an RFID reader;

in response to affirmative signals from the first and the second RFID tags, determining that the absorbent material is dry; and

in response to an affirmative signal from only the first RFID tag, determining that the absorbent material is wet.

C1. The method of C0, wherein the first RFID tag is disposed in a first zone of the incontinence product, and the second RFID tag is disposed in a second zone of the incontinence product.

C2. The method of C1, wherein the second zone corresponds to an area of the absorbent material expected to be wet when a user is incontinent.

C3. The method of C1 or C2, wherein the first zone corresponds to an area of the incontinence product expected to be dry when a user is incontinent.

C4. The method of C0, wherein the first RFID tag is configured to function in the presence of water.

C5. The method of C0, wherein the second RFID tag is configured to be detuned by the presence of water.

C6. The method of C5, wherein the first RFID tag is configured to be detuned by the presence of water.

C7. The method of C0, further comprising:

using the RFID reader, sending a signal to a remote server providing information regarding a moisture state of the incontinence product.

C8. The method of C7, further comprising:

in response to a determination that the absorbent material is wet, alerting a caregiver using the remote server.

C9. The method of C8, wherein alerting the caregiver comprises automatically sending a text message to an electronic device of the caregiver.

D0. A method for detecting moisture in an incontinence product, the method comprising:

providing an incontinence product comprising an absorbent material, a first UHF RFID tag, and a second UHF RFID tag;

interrogating the first and second RFID tags using an RFID reader; and

in response to an affirmative signal from only the first RFID tag, communicating an alert that the absorbent material is wet.

D1. The method of D0, wherein the alert is communicated by a server in communication with the RFID reader.

E0. A method for detecting moisture in an incontinence product, the method comprising:

interrogating, using an RFID reader, a first RFID tag and a second RFID tag, wherein the first and second RFID tags are included in an incontinence product; and

in response to an affirmative signal from only the first RFID tag, communicating an alert indicating that an absorbent material of the incontinence product is wet.

E1. The method of paragraph E0, wherein the alert is communicated by the RFID reader to a server.

E2. The method of paragraph E1, further comprising receiving, at a mobile digital device, a notification from the server based on the alert.

E3. The method of any one of paragraphs E0 through E2, wherein the first and second RFID tags comprise ultra-high frequency (UHF) RFID tags.

E4. The method of paragraph E3, wherein the first RFID tag is disposed in a first zone of the incontinence product expected to be dry when a user is incontinent, and the second RFID tag is disposed in a second zone of the incontinence product expected to be wet when a user is incontinent.

F0. A method for assessing wetness of an incontinence product including two or more RFID tags, the method comprising:

interrogating, using an RFID reader, the two or more RFID tags of the incontinence product; and

in response to affirmative signals from a number of the RFID tags, determining information about the wetness of the incontinence product.

F1. The method of paragraph F0, wherein the number is fewer than a total number of RFID tags of the incontinence product, and the information about the wetness of the incontinence product indicates that the incontinence product is wet.

F2. The method of any one of paragraphs F0 through F1, wherein determining information about the wetness of the incontinence product includes determining, based on the number, a degree of wetness of the incontinence product.

F3. The method of any one of paragraphs F0 through F2, wherein the two or more RFID tags include a first RFID tag configured to be detuned by the presence of water and a second RFID tag configured to function in the presence of water.

F4. The method of any one of paragraphs F0 through F2, wherein the two or more RFID tags comprise ultra-high frequency (UHF) tags.

### Advantages, Features, and Benefits

The different embodiments and examples of the moisture-sensing systems described herein provide several advantages over known solutions for detecting wetness in incontinence products. For example, illustrative embodiments and examples described herein allow an inexpensive moisture-sensing system using inexpensive components (e.g., standard UHF RFID tags).

Additionally, and among other benefits, illustrative embodiments and examples of RFID systems, devices, and methods described herein are configured to achieve passive, wireless monitoring of incontinence-related health conditions. This allows examples described herein to integrate seamlessly into, or even simplify existing care pathways, which is generally desirable for sensing products.

Additionally, and among other benefits, illustrative embodiments and examples described herein allow reliable, quick, and easy detection of moisture in incontinence products.

Additionally, and among other benefits, illustrative embodiments and examples described herein allow the wetness status of many incontinence products to be monitored, and allow wetness status notifications to be provided to caregivers and/or other appropriate parties.

Additionally, and among other benefits, illustrative embodiments and examples described herein allow historical data associated with wetness status of incontinence products to be stored and analyzed, facilitating identification of patterns and/or prediction of future needs.

No known system or device can perform these functions. However, not all embodiments and examples described herein provide the same advantages or the same degree of advantage.

### CONCLUSION

The disclosure set forth above may encompass multiple distinct examples with independent utility. Although each of these has been disclosed in its preferred form(s), the specific embodiments thereof as disclosed and illustrated herein are not to be considered in a limiting sense, because numerous variations are possible. To the extent that section headings are used within this disclosure, such headings are for organizational purposes only. The subject matter of the disclosure includes all novel and nonobvious combinations and subcombinations of the various elements, features, functions, and/or properties disclosed herein. The following claims particularly point out certain combinations and subcombinations regarded as novel and nonobvious. Other combinations and subcombinations of features, functions, elements, and/or properties may be claimed in applications claiming priority from this or a related application. Such claims, whether broader, narrower, equal, or different in scope to the original claims, also are regarded as included within the subject matter of the present disclosure.

1. A method for detecting moisture in an incontinence product, the method comprising:
interrogating, using a radio-frequency identification (RFID) reader, a first ultra-high frequency (UHF) RFID tag and a second UHF RFID tag, each of the first and second UHF RFID tags being coupled to an incontinence product comprising an absorbent material;
in response to affirmative signals from the first and the second RFID tags, determining that the absorbent material is dry; and
in response to an affirmative signal from only the first RFID tag, determining that the absorbent material is wet.

2. The method of claim 1, wherein the first UHF RFID tag is disposed in a first zone of the incontinence product, and the second UHF RFID tag is disposed in a second zone of the incontinence product.

3. The method of claim 2, wherein the second zone corresponds to an area of the absorbent material expected to be wet when a user is incontinent.

4. The method of claim 3, wherein the first zone corresponds to an area of the incontinence product expected to be dry when a user is incontinent.

5. The method of claim 1, wherein the first UHF RFID tag is configured to function in the presence of water.

6. The method of claim 1, wherein the second UHF RFID tag is configured to be detuned by the presence of water.

7. The method of claim 6, wherein the first UHF RFID tag is configured to be detuned by the presence of water.

8. The method of claim 1, further comprising:
sending information to a remote server using the RFID reader, wherein the information includes a moisture state of the incontinence product.

9. The method of claim 8, further comprising:
in response to a determination that the absorbent material is wet, alerting a caregiver using the remote server.

10. The method of claim 9, wherein alerting the caregiver comprises automatically sending a text message to an electronic device associated with the caregiver.

11. A method for detecting moisture in an incontinence product, the method comprising:
interrogating, using an RFID reader, a first RFID tag and a second RFID tag, wherein the first and second RFID tags are included in the incontinence product; and
in response to an affirmative signal from only the first RFID tag, communicating an alert indicating that an absorbent material of the incontinence product is wet.

12. The method of claim 11, wherein the alert is communicated by the RFID reader to a server.

13. The method of claim 12, further comprising receiving, at a mobile digital device, a notification from the server based on the alert.

14. The method of claim 11, wherein the first and second RFID tags comprise ultra-high frequency (UHF) RFID tags.

15. The method of claim 14, wherein the first RFID tag is disposed in a first zone of the incontinence product expected to be dry when a user is incontinent, and the second RFID tag is disposed in a second zone of the incontinence product expected to be wet when a user is incontinent.

16. A method for assessing wetness of an incontinence product including two or more RFID tags, the method comprising:
interrogating, using an RFID reader, the two or more RFID tags of the incontinence product; and
in response to affirmative signals from a number of the RFID tags, determining information about the wetness of the incontinence product.

17. The method of claim 16, wherein the number is fewer than a total number of RFID tags of the incontinence product, and the information about the wetness of the incontinence product indicates that the incontinence product is wet.

18. The method of claim 16, wherein determining information about the wetness of the incontinence product includes determining, based on the number, a degree of wetness of the incontinence product.

19. The method of claim 16, wherein the two or more RFID tags include a first RFID tag configured to be detuned by the presence of water and a second RFID tag configured to function in the presence of water.

20. The method of claim 16, wherein the two or more RFID tags comprise ultra-high frequency (UHF) tags.
